(19) United States
(12) Patent Application Publication
(10) Pub. No.: US 2026/0232365 A1
(43) Pub. Date: Aug. 13, 2026

(54) END EFFECTOR WITH TISSUE COMPRESSION FEATURES AND METHODS OF USING THE SAME

(71) Applicant: Cilag GmbH International, Zug (CH)

(72) Inventors: Steven P. Smolik, West Chester, OH (US); Eric W. Brunner, Cincinnati, OH (US)

(21) Appl. No.: 19/052,518

(22) Filed: Feb. 13, 2025

(51) Int. Cl. A61B 18/00

(52) U.S. Cl. CPC A61B 18/00; A61B 2018/00083; A61B 2018/00136; A61B 2018/00184; A61B 2018/00601; A61B 2018/00994

(57) ABSTRACT

A surgical instrument, comprising: a shaft assembly; and an end effector extending distally from the shaft assembly, wherein the end effector includes an electrode configured to apply energy to a tissue of a patient, the electrode comprising: an electrode base surface, and a patterned coating layer having a coating material on the electrode base surface defining a raised profile offset from the electrode base surface, wherein the patterned coating layer further defines a plurality of openings to thereby expose a plurality of bare electrode portions of the electrode base surface therethrough, wherein a coverage ratio of the raised profile of the patterned coating layer to the plurality of bare electrode portions of the electrode base surface is between about 15% to about 75% of a total surface area of the electrode base surface.

### BACKGROUND

A variety of surgical instruments include a tissue cutting element and one or more elements that transmit radio frequency (RF) energy to tissue (e.g., to coagulate or seal the tissue). An example of such an electrosurgical instrument is the ENSEAL® Tissue Sealing Device by Ethicon Endo-Surgery, Inc., of Cincinnati, Ohio. Other examples of such devices and related concepts are disclosed in U.S. Pat. No. 7,354,440, entitled “Electrosurgical Instrument and Method of Use,” issued Apr. 8, 2008, the disclosure of which is incorporated by reference herein; U.S. Pat. No. 7,381,209, entitled “Electrosurgical Instrument,” issued Jun. 3, 2008, the disclosure of which is incorporated by reference herein.

Some instruments are capable of applying both ultrasonic energy and RF electrosurgical energy to tissue. Examples of such instruments are described in U.S. Pat. No. 9,949,785, entitled “Ultrasonic Surgical Instrument with Electrosurgical Feature,” issued Apr. 24, 2018, the disclosure of which is incorporated by reference herein; and U.S. Pat. No. 8,663,220, entitled “Ultrasonic Electrosurgical Instruments,” issued Mar. 4, 2014, the disclosure of which is incorporated by reference herein.

U.S. Pat. No. 9,272,095, entitled “Vessels, Contact Surfaces, and Coating and Inspection Apparatus and Methods,” issued on Mar. 1, 2016, relates to fabrication of coated contact surfaces of a medical device. U.S. Pat. No. 9,272,095 describes one utility for such a hydrophobic layer is to isolate a thermoplastic tube wall, made for example of polyethylene terephthalate (PET), from blood collected within the tube. A hydrophobic layer can be applied on top of a hydrophilic SiO₂, coating on the internal contact surface of the tube and the hydrophobic layer precursor can comprise hexamethyldisiloxane (HMDSO) or octamethylcyclotetrasiloxane (OMCTS). U.S. Pat. No. 9,272,095 does not appear to disclose hydrophobic coating being applied in addition to at least one of the microscopic surface pattern or the nanoscopic surface roughness.

U.S. Pub. No. 2014/0276407, entitled “Medical Devices Having Micropatterns,” published on Sep. 14, 2014, now abandoned, describes a plurality of nanostructures, a plurality of microstructures, and a plurality of hierarchical structures. A micropatterned polymer coating may be formed of any suitable material for a particular application, and may include one or more of a flexible polymer, a rigid polymer, a metal, an alloy, and any other material that may be suitable for a particular application. The micropatterned polymer coating could be applied by any of a wide variety of manufacturing techniques described herein including extrusion, compression dies, electro deposition, photoetching, or over molding configurations. U.S. Pub. No. 2014/0276407 does not appear to disclose a hydrophobic coating being applied in addition to at least one of the microscopic surface pattern or the nanoscopic surface roughness.

U.S. Pub. No. 2013/0138103 entitled “Electrosurgical Unit with Micro/nano Structure and the Manufacturing Method Thereof,” published on May 30, 2013, now abandoned, describes in FIG. 2 using the irradiation of the laser beam to directly construct a micro/nano structure on the surface of the blade while allowing the micro/nano structure to be composed of a hybrid of micro/nano elements. Referring to FIG. 3, the micro/nano structure 13 is formed directly on the blade 11. U.S. Pub. No. 2013/0138103 does not appear to disclose a hydrophobic coating in addition to the micro/nano structure.

U.S. Pat. No. 11,497,546 entitled “Area ratios of patterned coatings on RF electrodes to reduce sticking”, issued on Nov. 15, 2022, relates to an electrosurgical system includes an RF current generator, a handle body, and an end effector, wherein at least a portion of either a first or second energy delivery surface of the end effector, or both, may include one or more patterned coatings of an electrically non-conducting non-stick material. U.S. Pat. No. 11,497,546 teaches that the patterned coating may be formed from a coating of a material from which portions have been removed. U.S. Pat. No. 11,497,546 does not appear to disclose utilizing laser technology to remove portions of the coating material.

While several surgical instruments and systems have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary electrosurgical instrument;

FIG. 2 depicts a perspective view of an exemplary articulation assembly and end effector of the electrosurgical instrument of FIG. 1;

FIG. 3 depicts an exploded view of the articulation assembly and end effector of FIG. 2;

FIG. 4 depicts a perspective view of the end effector that of FIG. 2;

FIG. 5 depicts an exploded perspective view of the end effector of FIG. 2;

FIG. 6 depicts a top view of a jaw wherein a coating material has been applied and forms a crisscross patterned formation;

FIG. 7 depicts a top view of a jaw wherein a coating material has been applied and forms a honeycomb patterned formation

FIG. 8 depicts a cross-sectional view of a coating material applied and then selectively removed to form a patterned formation with removed portions having a removal depth of less than an entirety of a coating application thickness of the applied coating material;

FIG. 9 depicts a cross-sectional view of a coating material applied and then selectively removed to form a patterned formation with removed portions having a removal depth equal to an entirety of a coating application thickness of the applied coating material;

FIG. 10 depicts a side view of a jaw wherein a coating material has been applied and form a patterned formation wherein a coating application thickness of the applied coating material is varied along a length of the jaw.

The drawings are not intended to be limiting in any way, and it is contemplated that various versions of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, versions, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers to the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. In addition, the terms “upper,” “lower,” “top,” and “bottom,” are used with respect to the examples and associated figures and are not intended to unnecessarily limit the invention described herein.

### I. Exemplary of Electrosurgical Instrument

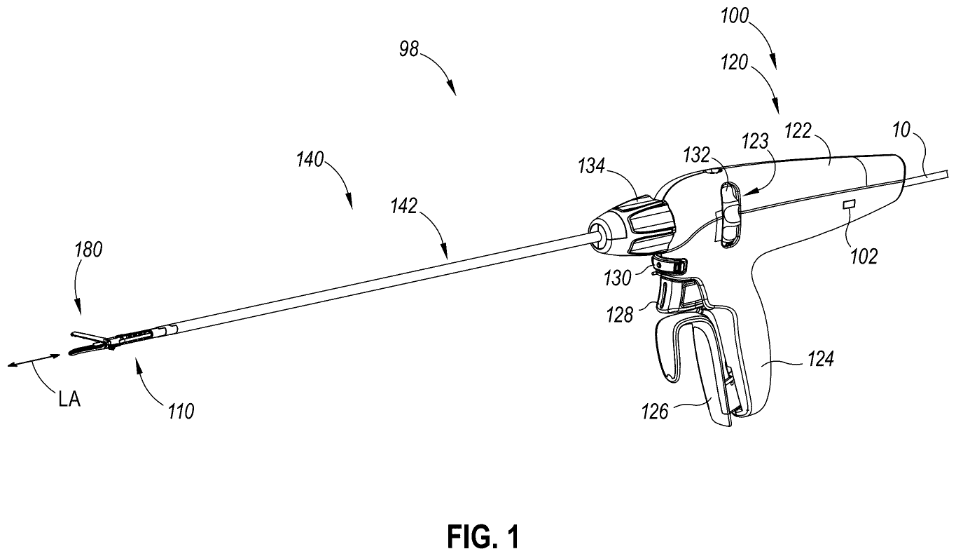

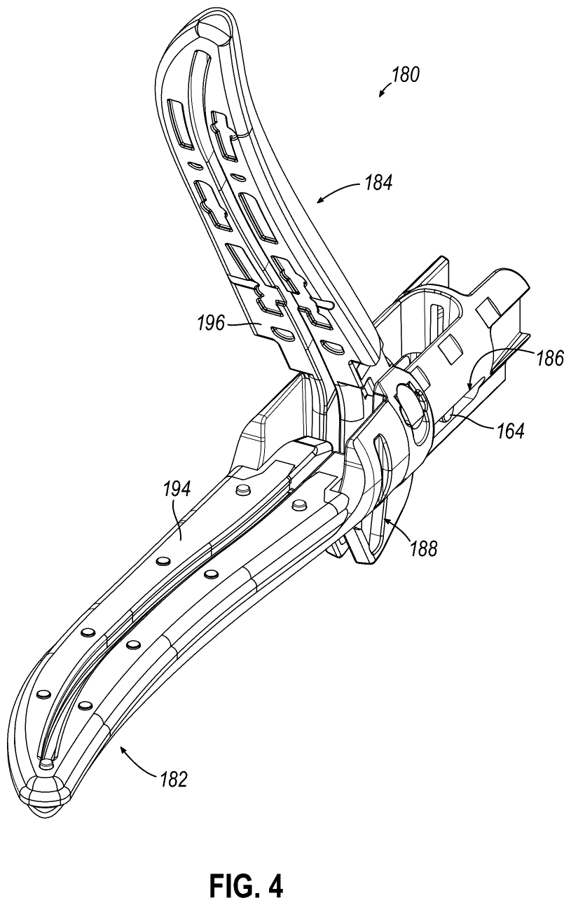

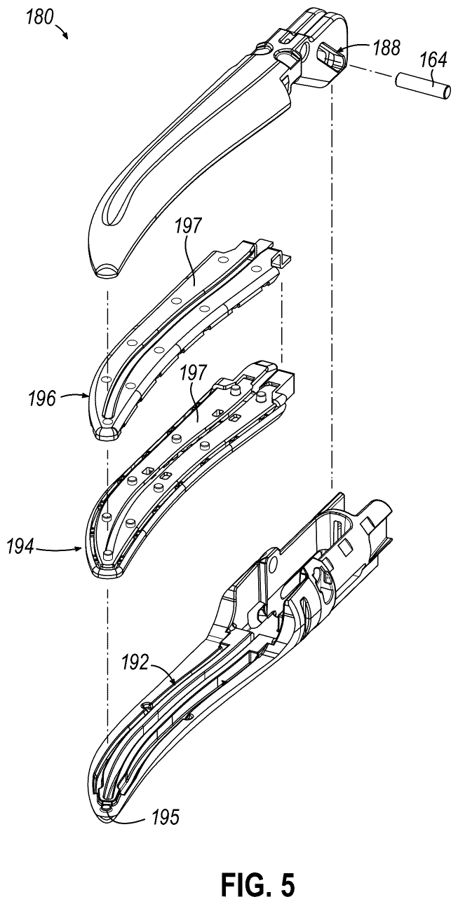

FIGS. 1-5 show a surgical system (98) including an exemplary electrosurgical instrument (100). As best seen in FIG. 1, electrosurgical instrument (100) includes a handle assembly (120), a shaft assembly (140), an articulation assembly (110), which may also be referred to as an articulation section (110), and an end effector (180). As will be described in greater detail below, end effector (180) of electrosurgical instrument (100) may be operable to grasp, cut, and seal or weld tissue (e.g., a blood vessel, etc.). In this example, end effector (180) may be configured to apply a non-therapeutic bipolar radio frequency (RF) energy in order to identify and/or verify that the correct tissue may be present in the end effector such that a therapeutic RF energy can be applied to seal or weld tissue. However, it should be understood that electrosurgical instrument (100) may be configured to seal or weld tissue through any other suitable means that would be apparent to one skilled in the art in view of the teachings herein. For example, electrosurgical instrument (100) may be configured to seal or weld tissue via an ultrasonic blade, staples, etc. In the present example, electrosurgical instrument (100) may be electrically coupled to a waveform generator (200) of surgical system (98), which may be capable of delivering therapeutic and non-therapeutic energy, via power cable (10).

Waveform generator (200) may be configured to provide all or some of the electrical power requirements for use of electrosurgical instrument (100). Any suitable waveform generator (200) may be used as would be apparent to one skilled in the art in view of the teachings herein. By way of non-limiting example, the waveform generator (200) may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein, in its entirety. While in the current example, electrosurgical instrument (100) may be coupled to waveform generator (200) via power cable (10), electrosurgical instrument (100) may contain an internal power source or plurality of power sources, such as a battery and/or supercapacitors, to electrically power electrosurgical instrument (100). Of course, any suitable combination of power sources may be utilized to power electrosurgical instrument (100) as would be apparent to one skilled in the art in view of the teaching herein.

Handle assembly (120) may be configured to be grasped by an operator with one hand, such that an operator may control and manipulate electrosurgical instrument (100) with a single hand. Although electrosurgical instrument (100) may be primarily described herein as being used by a human user, it should be noted that alternative versions exist in which one or more robotic systems (e.g., a robotic arm) may be used to control and manipulate electrosurgical instrument (100). Shaft assembly (140) extends distally from handle assembly (120) and connects to articulation assembly (110). Articulation assembly (110) may also be connected to a proximal end of end effector (180). As will be described in greater detail below, components of handle assembly (120) may be configured to control end effector (180) such that an operator may grasp, cut, and seal or weld tissue. Articulation assembly (110) may be configured to deflect end effector (180) from the longitudinal axis (LA) defined by shaft assembly (140).

Handle assembly (120) of the present example includes a control unit (102) housed within a body (122), a pistol grip (124), a jaw closure trigger (126), a knife trigger (128), an activation button (130), an articulation control (132), and a knob (134). As will be described in greater detail below, jaw closure trigger (126) may be pivoted toward and away from pistol grip (124) and/or body (122) to open and close jaws (182, 184) of end effector (180) to grasp tissue. Additionally, knife trigger (128) may be pivoted toward and away from pistol grip (124) and/or body (122) to actuate a knife member (176) within the confines of jaws (182, 184) to cut tissue captured between jaws (182, 184). Further, activation button (130) may be pressed to apply radio frequency (RF) energy to tissue via electrodes (194, 196) of jaws (182, 184), respectively. In some versions, electrodes (194, 196) of jaws (182, 184) may be in a bifurcation configuration where electrodes (194, 196) move relative to a central axis and nearly equal and opposite to one another.

Body (122) of handle assembly (120) defines an opening (123) through which a portion of articulation control (132) protrudes. Articulation control (132) may be rotatably disposed within body (122) such that an operator may rotate the portion of articulation control (132) protruding from opening (123) to rotate the portion of articulation control (132) located within body (122). Rotation of articulation control (132) relative to body (122) may bend articulation assembly (110) in order to drive deflection of end effector (180) from the longitudinal axis (LA) defined by shaft assembly (140). Articulation control (132) and articulation assembly (110) may include any suitable features to drive deflection of end effector (180) from the longitudinal axis (LA) defined by shaft assembly (140) as would be apparent to one skilled in the art in view of the teachings herein.

Knob (134) may be rotatably disposed on the distal end of body (122) and may be configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) may be rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

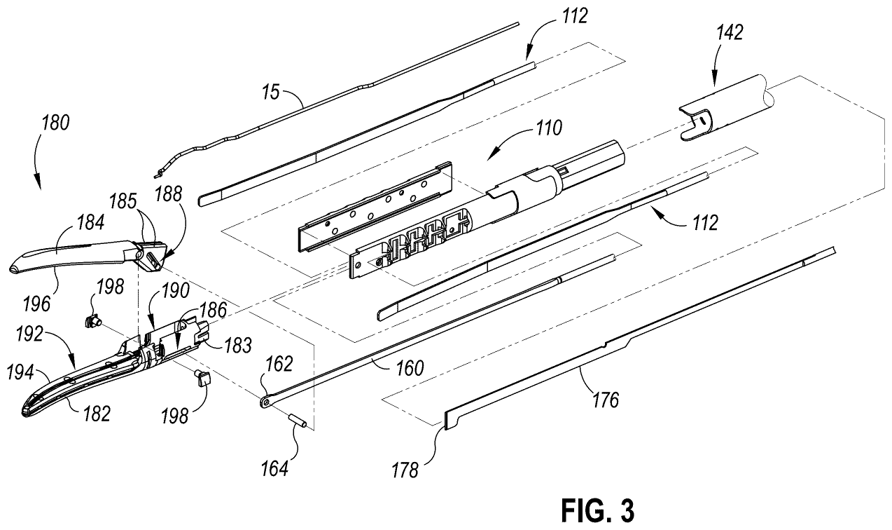

Shaft assembly (140) may include distal portion (142) extending distally from handle assembly (120) and a proximal portion housed within the confines of body (122) of handle assembly (120). Referring to FIG. 3, shaft assembly (140) houses a jaw closure connector (160) that couples jaw closure trigger (126) with end effector (180). Additionally, shaft assembly (140) houses a portion of knife member (176) extending between a distal cutting edge (178) of knife member (176) and knife trigger (128). Shaft assembly (140) also houses actuating members (112) that couple articulation assembly (110) with articulation control (132); as well as an electrical coupling (15) that operatively couples electrodes (194, 196) with activation button (130). As will be described in greater detail below, jaw closure connector (160) may be configured to translate relative to shaft assembly (140) to open and close jaws (182, 184) of end effector (180); while knife member (176) may be coupled to knife trigger (128) of handle assembly (120) to translate distal cutting edge (178) within the confines of end effector (180); and activation button (130) may be configured to activate electrodes (194, 196).

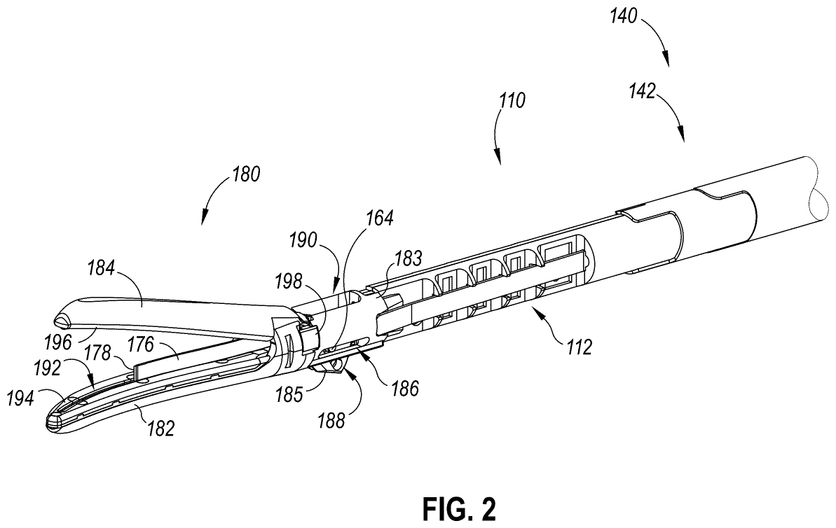

As best seen in FIGS. 2-5, end effector (180) includes lower jaw (182) pivotally coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that may be configured to receive proximal arms (185) of upper jaw (184), portions of knife member (176), jaw closure connector (160), and pin (164). Slots (186, 188) each slidably receive pin (164), which is attached to a distal coupling portion (162) of jaw closure connector (160). Additionally, lower jaw (182) includes a force sensor (195) located at a distal tip of lower jaw (182), though force sensor (195) may alternatively be positioned at any other suitable location. Force sensor (195) may be in communication with control unit (102). Force sensor (195) may be configured to measure the closure force generated by pivoting jaws (182, 184) into a closed configuration in accordance with the description herein. Additionally, force sensor (195) may communicate this data to control unit (102). Any suitable components may be used for force sensor (195) as would be apparent to one skilled in art in view of the teachings herein. For example, force sensor (195) may take the form of a strain gauge. In some variations, end effector (180) includes more than one force sensor.

While in the current example, a force sensor (195) may be incorporated into electrosurgical instrument (100) and may be in communication with control unit (102), any other suitable sensors or feedback mechanisms may be additionally or alternatively incorporated into electrosurgical instrument (100) while in communication with control unit (102) as would be apparent to one skilled in the art in view of the teachings herein. For instance, an articulation sensor or feedback mechanism may be incorporated into electrosurgical instrument (100), where the articulation sensor may communicate signals to control unit (102) indicative of the degree end effector (180) may be deflected from the longitudinal axis (LA) by articulation control (132) and articulation assembly (110).

As will be described in greater detail below, jaw closure connector (160) may be operable to translate within central channel (190) of lower jaw (182). Translation of jaw closure connector (160) may drive pin (164). As will also be described in greater detail below, with pin (164) being located within both slots (186, 188), and with slots (186, 188) being angled relative to each other, pin (164) cams against proximal arms (185) to pivot upper jaw (184) toward and away from lower jaw (182) about pivot couplings (198). Therefore, upper jaw (184) may be configured to pivot toward and away from lower jaw (182) about pivot couplings (198) to grasp tissue.

The term “pivot” does not necessarily require rotation about a fixed axis and may include rotation about an axis that moves relative to end effector (180). Therefore, the axis at which upper jaw (184) pivots about lower jaw (182) may translate relative to both upper jaw (184) and lower jaw (182). Any suitable translation of the pivot axis may be used as would be apparent to one skilled in the art in view of the teachings herein.

Lower jaw (182) and upper jaw (184) may also define a knife pathway (192). Knife pathway (192) may be configured to slidably receive knife member (176), such that knife member (176) may be retracted, and advanced, to cut tissue captured between jaws (182, 184).

Lower jaw (182) and upper jaw (184) may each comprise respective electrodes (194, 196). The power source may provide RF energy to electrodes (194, 196) via electrical coupling (15) that extends through handle assembly (120), shaft assembly (140), articulation assembly (110), and electrically couples with one or both of electrodes (194, 196). Electrical coupling (15) may selectively activate electrodes (194, 196) in response to an operator pressing activation button (130). In some instances, control unit (102) may couple electrical coupling (15) with activation button (130), such that control unit (102) activates electrodes (194, 196) in response to operator pressing activation button (130). Control unit (102) may have any suitable components in order to perform suitable functions as would be apparent to one skilled in the art in view of the teachings herein. For instance, control unit (102) may have a processor, memory unit, suitable circuitry, etc. Examples of features and functionalities that may be incorporated into control unit (102) will be described in greater detail below.

As described above, jaw closure trigger (126) may be pivoted toward and away from pistol grip (124) and/or body (122) to open and close jaws (182, 184) of end effector (180) to grasp tissue. In particular, as will be described in greater detail below, pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

In some versions, knife trigger (128) may be pivoted toward and away from body (122) and/or pistol grip (124) to actuate knife member (176) within knife pathway (192) of jaws (182, 184) to cut tissue captured between jaws (182, 184). In particular, handle assembly (120) further includes a knife coupling body that may be slidably coupled along proximal portion of shaft assembly (140). Knife coupling body may be coupled with knife member (176) such that translation of knife coupling body relative to proximal portion of shaft assembly (140) translates knife member (176) relative to shaft assembly (140).

In another version, knife coupling body may be coupled to a knife actuation assembly such that as knife trigger (128) pivots toward body (122) and/or pistol grip (124), knife actuation assembly drives knife coupling body distally, thereby driving knife member (176) distally within knife pathway (192). Because knife coupling body may be coupled to knife member (176), knife member (176) translates distally within shaft assembly (140), articulation assembly (110), and within knife pathway (192) of end effector (180). Knife member (176) includes distal cutting edge (178) that may be configured to sever tissue captured between jaws (182, 184). Therefore, pivoting knife trigger (128) causes knife member (176) to actuate within knife pathway (192) of end effector (180) to sever tissue captured

### Between Jaws (182, 184).

With distal cutting edge (178) of knife member (176) actuated to the advanced position, an operator may press activation button (130) to selectively activate electrodes (194, 196) of jaws (182, 184) to seal or weld severed tissue captured between jaws (182, 184). It should be understood that the operator may also press activation button (130) to selectively activate electrodes (194, 196) of jaws (182, 184) at any suitable time during exemplary use. Therefore, the operator may also press activation button (130) while knife member (176) may be retracted. Next, the operator may release jaw closure trigger (126) such that jaws (182, 184) pivot into the opened configuration, releasing tissue.

### II. Exemplary Deposition of Dielectric Material

### A. Overview

Instruments, such as instrument (100) may generate heat as end effectors, such as end effector (180), seal and/or cut tissue. Tissue contacting surfaces of the instruments may tend to stick to the treated tissue. The tissue contacting surfaces are intended to include at least one of an ultrasonic blade, electrodes (194, 196), or another suitable design. The tissue contacting surfaces include an electrode base surface (197) that is configured to contact the tissue. For example, electrode base surface (197) may include, for example, an outer surface of an ultrasonic blade or an electrode surface of electrodes (194, 196). Tissue sticking may cause reduced surgical efficiency.

The issue of tissue sticking is typically overcome by the addition of a coating layer to the tissue contacting surfaces of an instrument. However, the addition of a coating layer, which is typically electrically insulative, to the tissue contacting surfaces limits the generation of the electrical field and consequently the work done to the tissue. One of the byproducts of the limited generation of the electrical field may be that the tissue cut by the instrument has low burst pressure. To improve the electrical current transfer between tissue contacting surfaces while maintaining tissue anti-sticking performance, the present disclosure applies a dielectric material in a patterned formation, which may also be referred to herein as a patterned coating layer. The presence of the alternating current electrical field may heat the targeted tissue. The heat may then cause the collagen and elastin within the tissue to coagulate and fuse. The coating, acting as insulator, could lessen this affect and lower burst pressure.

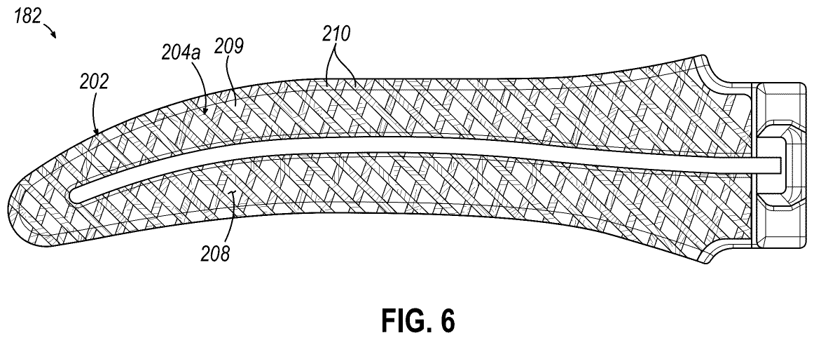

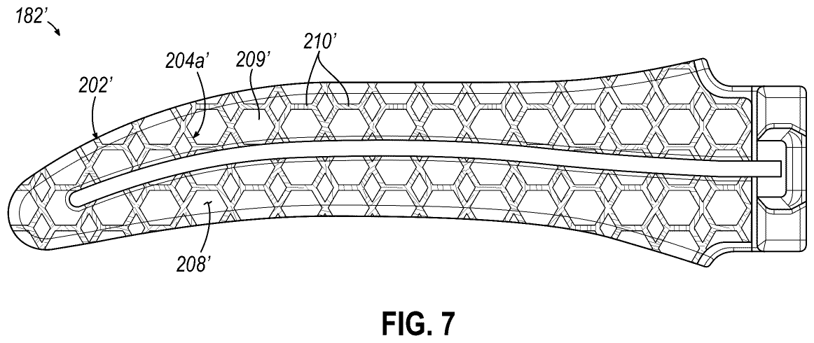

As will be described in greater detail below with reference to FIGS. 6-14, tissue contacting surfaces, such as tissue contacting surface (208) may include an applied coating material (202) directly applied or later formed into a patterned formation (204) to reduce sticking or otherwise promote tissue release while also improving the electrical current transfer between tissue contacting surfaces without substantially impeding the electrical current transfer. Specifically, FIGS. 6 and 7 show lower jaw (182) of exemplary electrosurgical instrument (100) wherein coating material (202) has been directly applied or later formed into patterned formation (204) on tissue contacting surface (208). Although FIGS. 6 and 7 both show lower jaw (182), upper jaw (184) can have coating material (202) that has been directly applied or later formed into patterned formation (204) on tissue contacting surface (208) of upper jaw (184). Such patterned formation (204) defines a plurality of openings (209) exposing a plurality of bare electrode portions (210) of electrode base surface (197) of one or more electrodes (194, 196). While patterned formations (204) are described with reference to being applied to tissue contacting surfaces (208) of electrodes (194, 196), patterned formations (204) can also be formed on an ultrasonic blade or another suitable surface that receives a coating layer. As previously described, electrodes (194, 196) may be configured to cooperate to apply bipolar RF energy to tissue.

It is envisioned that patterned formations (204) may be applied to select portions of the tissue contacting surfaces (208). Alternatively, patterned formations (204) may be applied to the entire tissue contacting surface (208). In some versions, patterned formations (204) may be applied to the entire electrode base surface (197) of electrodes (194, 196) or the entire electrode base surface (197) of an ultrasonic blade. In other versions, patterned formations (204) may be applied to only select electrode base surfaces (197) of electrodes (194, 196) or to select electrode base surfaces of an ultrasonic blade that experience sticking or high-pressure during tissue clamping. Patterned formations (204) may be disposed on a metallic surface of the tissue contacting surface.

### B. Coating Layer

In one or more versions, coating material (202) includes a dielectric material. The present disclosure defines the term dielectric material as being an insulating material that does not conduct electricity, but that can support an electrostatic field. In one or more versions, dielectric materials that can be utilized as coating material (202) may include plastics such as polyethylene or polystyrene; ceramics such as barium titanate, alumina, zirconia, or silicon nitride; polymers such as silicon resins, epoxies, polyimides, or polycarbonates; glass materials such as borosilicate glass, solder glass, or sealing glass; and combinations thereof.

### C. Application of the Coating Layer

In one or more versions, coating material (202) can be applied via a method that applies coating material (202) directly into patterned formation (204). Such application methods may include injection molding, insert molding, overmolding, lamination, screen printing, die bonding, spray coating, dip coating, spin coating, or additive manufacturing. After application of coating material (202) into patterned formation (204), there may be the desired spacing between electrodes while ensuring adequate current flow to achieve the desired tissue effect.

In one or more versions, coating material (202) can be applied via a method that coats the entire surface, such as the entire surface (208) of electrodes (194, 196), then portions of applied coating material (202) can be selectively removed to form patterned formation (204). In one or more versions, the selective removal may be accomplished through laser ablation, photolithographic removal, acid etching, chemical etching, plasma etching, abrasive spraying, or combinations thereof. After selective removal of coating material (202) into patterned formation (204), there may be a desired spacing between electrodes while ensuring adequate current flow to achieve the desired tissue effect.

### D. Pattern Formation

To address the low burst performance, coating material (202) can be directly applied or later formed into patterned formation (204). In one or more versions, the utilization of patterned formation (204) can allow for the RF energy to pass through or be less impacted by coating material (202) and as a result, the burst performance of a seal formed by instrument (100) containing coating material (202) in patterned formation (204) is improved as compared to the burst performance of a seal formed by instrument (100) containing coating material (202) not utilizing patterned formation (204). However, there is fine balance between improving the burst performance while maintaining the tissue anti-sticking performance of applied coating material (202).

Patterned formation (204) may optimize compressive strength while minimizing the dielectric coverage area on the surface it is applied to. Patterned formation (204) may include, but are not limited to, shapes, such as squares, rectangles, circles, triangles, and/or hexagons (honeycomb). The purpose of patterned formation (204) may be to ensure necessary gaps, such as openings (209), while facilitating optimal load distribution throughout patterned formation (204). Furthermore, effective pressure dissipation requires strategically designed load paths to reduce stress concentrations and maintain sufficient structural integrity to prevent loss of gap across the entire surface. This optimized strength-to-weight design may also be crucial for allowing electrical field transmission through exposed sections, such as openings (209) and providing adequate spacing to accommodate tissue without excessively compressing/damaging it.

Although the present disclosure is not limited by the specific pattern or formation (204), FIG. 6 shows an example of a crisscross pattern (204a), and FIG. 7 shows an example of a honeycomb pattern (204a′) incorporated into an alternative lower jaw (182′), which is like lower jaw (182) unless otherwise specifically discussed herein. Honeycomb pattern (204a′) provides outstanding compressive strength to the coating material (202′). Higher pressures on coating material (202′) are expected as there may be an expected clamping force applied to the jaws. Higher pressure may be expected due to their smaller area exposure, and the force applied to smaller areas therefore results in higher pressure. Similar to FIG. 6, the honeycomb pattern (204a′) shown in FIG. 7 defines a plurality of openings (209′) exposing a plurality of bare electrode portions (210′). The openings (209′) may decrease the amount of tissue sticking compared to base substrates (208′) having generally smooth surfaces. This outstanding compressive strength of honeycomb pattern (204a′) may arise from several factors. The hexagonal cell configuration of honeycomb pattern (204a′) may allow for efficient load distribution, ensuring that forces are spread evenly, minimizing localized peaks of stress. The walls of the cells of honeycomb pattern (204a′) can carry the load evenly if loaded properly. Additionally, the geometry of the thin, interconnected cell walls of honeycomb pattern (204a′) may enhance resistance to buckling under compressive loads, as the interconnected cell walls of honeycomb pattern (204a′) can redistribute stress while maintaining stability. The optimal thickness of the interconnected cell walls of honeycomb pattern (204a′) may be essential for achieving maximum compressive strength without adding unnecessary weight. Furthermore, honeycomb structures, such as honeycomb pattern (204a′) naturally exhibit energy absorption capabilities; when subjected to compressive forces, they experience a progressive collapse that absorbs energy through plastic deformation, which aids in dissipating impact energy.

### E. Characteristics of Patterned Formation

By controlling the size and depth of patterned formation (204, 204′) relative to base substrate (208, 208′), plurality of openings (209, 209′) exposing the plurality of bare electrode portions (210, 210′) may decrease the amount of tissue sticking compared to base substrates (208, 208′) having generally smooth surfaces. For example, plurality of openings (209, 209′) exposing the plurality of bare electrode portions (210, 210′) may reduce tissue sticking compared to base substrates (208, 208′), which may reduce the number of protein bonding sites.

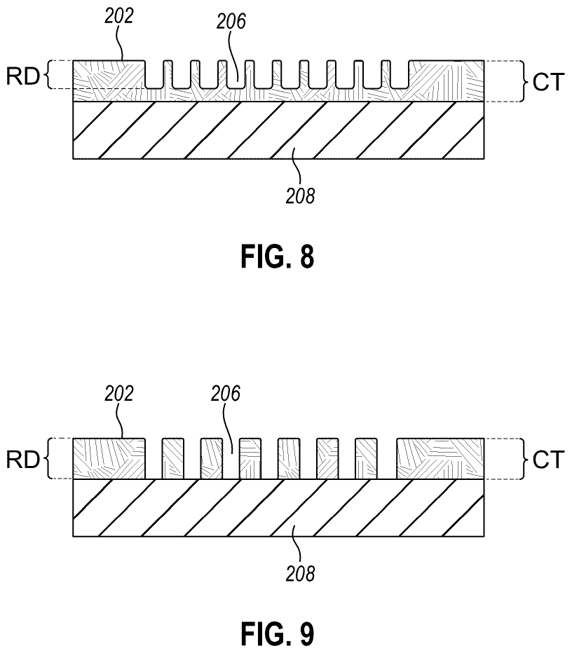

Patterned formations (204a, 204a′) shown in FIGS. 6 and 7 each contain a coating layer thickness (CT) of the applied coating material (202, 202′) that makes up patterned formations (204a, 204a′). In instances wherein coating material (202, 202′) can be applied via a method that coats the entire surface, such as the entire surface (208, 208′), then portions of applied coating material (202, 202′) can be selectively removed to form patterned formation (204a, 204a′), plurality of openings (209, 209′) exposing the plurality of bare electrode portions (210, 210′) may be removed such that plurality of openings (209, 209′) have a removal depth (RD) of less than the entirety of a coating application thickness (CT) of applied coating material (202, 202′) such that each plurality of openings (209, 209′) does not reach down to substrate (208, 208′) of lower jaw (182), such as shown in FIG. 8. In yet other versions, plurality of openings (209, 209′) have a removal depth (RD) equal to coating application thickness (CT) such that portions plurality of openings (209, 209′) reach down to substrate (208, 208′), such as shown in FIG. 9. In one or more versions, removal depth (RD) of plurality of openings (209, 209′) may be from about 50% to about 100% of coating application thickness (CT), from about 50% to about 80%, from about 50% to about 70%, and from about 50% to about 60%. In one or more versions, removal depth (RD) of plurality of openings (209, 209′) can have any value between any of the foregoing ranges.

In instances wherein coating material (202, 202′) can be applied via a method that coats the entire surface, such as the entire surface (208, 208′), then portions of applied coating material (202, 202′) can be selectively removed to form patterned formation (204a, 204 a′), from about 60% to about 85% of applied coating material (202, 202′) can be removed to form plurality of openings (209, 209′) and patterned formation (204a, 204a′). In other versions, from about 65% to about 80% of applied coating material (202, 202′) can be removed to form plurality of openings (209, 209′) and patterned formation (204a, 204a′). In yet other versions, from about 50% to about 75% of applied coating material (202, 202′) can be removed to form plurality of openings (209, 209′) and patterned formation (204a, 204a′). In one or more versions, the percentage of applied coating material (202, 202′) removed can have any value between any of the foregoing ranges. In one or more versions, the percentage of applied coating (202, 202′) removed to form plurality of openings (209, 209′) can be adjusted by increasing the number of openings (209, 209′), by increasing removal depth (RD) of each opening (209, 209′), by increasing the diameter of each opening (209, 209′), or combinations thereof.

In instances wherein coating material (202, 202′) can be applied via a method that coats the entire surface, such as the entire surface (208, 208′), then portions of applied coating material (202, 202) can be selectively removed to form patterned formation (204a, 204a′), although plurality of openings (209, 209′) as shown in FIGS. 6 and 7 have been removed to form patterned formation (204a, 204a′), in yet other versions plurality of openings (209, 209′) can be removed in an un-patterned formation.

In one or more versions, coating material (202, 202′) forming patterned formation (204 a, 204 a′) can have coating application thickness (CT) of between about 150 nm to 700 nm, 250 nm and about 600 nm, between about 350 nm and about 500 nm, or between about 400 nm and about 450 nm. In one or more versions, coating material (202, 202′) can have coating application thickness (CT) between any of the foregoing ranges.

In one or more versions, coating material (202, 202′) forming patterned formation (204 a, 204 a′) can have a coverage ratio over surface (208, 208′) of between about 15% and about 75%, in other versions from about 20% to about 60%, and in yet other versions from about 25% to about 40%. In one or more versions, the coverage ratio over surface (208, 208′) can have any value between any of the foregoing ranges.

In one or more versions, coating application thickness (CT) of coating material (202, 202′) and configuration of patterned formation (204a, 204a′) may be varied to ensure that a sufficient electrical field may be generated for the target tissue. Adjustments to the height, placement, and density of coating material (202, 202′) forming patterned formation (204a, 204a′) could be made to produce the intended tissue response while reducing the likelihood of electrical shorting due to inadequate gapping. The method of applying coating material (202, 202′) to form patterned formation (204, 204′) also facilitate appropriate pressure distribution between the two active electrode plates. Optimization of a location, height, and density of patterned formation (204a, 204a′) could be based on the clamping pressure measured across the entire parallel plate surfaces. For instance, honeycomb structure (204a′) may serve as a minimal density design that allows for effective electrical field generation while preserving relatively high out-of-plane compression characteristics. Additionally, considerations regarding the height, placement, and density of coating material (202, 202′) forming patterned formation (204a, 204a′) may take into consideration local compressive forces when the electrodes, such as electrodes (194, 196), are closed together, allowing patterned formation (204a, 204a′) to adapt to the observed force distribution.

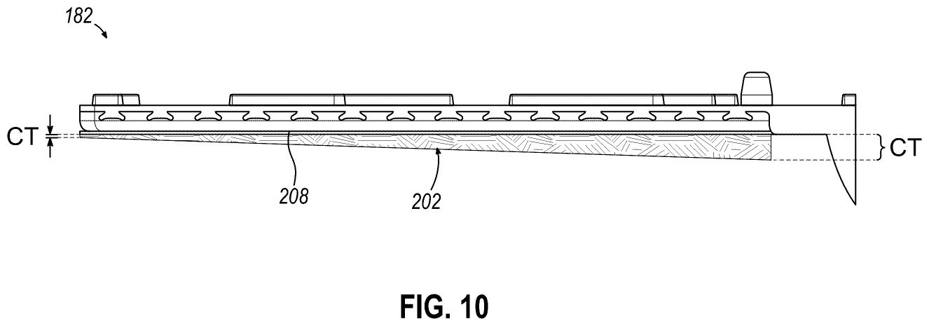

In one or more versions, such as shown in FIG. 10, it is contemplated that coating application thickness (CT) of coating material (202) is varied along a length of lower jaw (182). Although FIG. 10 shows lower jaw (182) only, in instances wherein coating application thickness (CT) of coating material (202) is varied, upper jaw (184) can have coating application thickness (CT) of coating material (202) that is varied in a complementary manner to the variedness of coating material (202) on lower jaw (182). As shown in FIG. 10, coating application thickness (CT) at a distal end (DE) of lower jaw (182) is less than the coating application thickness (CT) at a proximal end (PE) of lower jaw (182). Having a coating application thickness (CT) of coating material (202) varied along a length of lower jaw (182) may allow for patterned formation (204) to achieve certain gap and tissue effects. Modifying the coating application thickness (CT) of coating material (202) could allow for gap control to be designed relative to the applied clamping force and bending of the opposing jaws. The modified coating application thickness (CT) of coating material (202) may be designed to provide necessary gap control for this anticipated phenomenon.

Aspects of the patterns (204a, 204a′) are not necessarily distinct to each other and may be incorporated in whole or in part into alternative patterns (204). The invention is therefore not intended to be unnecessarily limited to patterns (204a, 204a′) as shown in the present examples.

### II. Illustrative Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A surgical instrument comprising: a shaft assembly; and an end effector extending distally from the shaft assembly, wherein the end effector includes an electrode configured to apply energy to a tissue of a patient, the electrode comprising: an electrode base surface; and a patterned coating layer having a coating material on the electrode base surface defining a raised profile offset from the electrode base surface, wherein the patterned coating layer further defines a plurality of openings to thereby expose a plurality of bare electrode portions of the electrode base surface therethrough, wherein a coverage ratio of the raised profile of the patterned coating layer to the plurality of bare electrode portions of the electrode base surface is between about 15% to about 75% of a total surface area of the electrode base surface.

### EXAMPLE 2

The surgical instrument of Example 1, wherein the coating material includes a dielectric material.

### EXAMPLE 3

The surgical instrument of Example 1 or 2, wherein the dielectric material includes at least one of a plastics, a ceramic, a polymer, or a glass material.

### EXAMPLE 4

The surgical instrument of one or more of Example 1 through 3, wherein the patterned coating layer includes a coating application thickness of between about 150 nm and about 700 nm.

### EXAMPLE 5

The surgical instrument of one or more of Example 1 through 4, wherein the patterned coating layer has a coating application thickness that is consistent across the entirety of the raised profile over the electrode base surface.

### EXAMPLE 6

The surgical instrument of one or more of Example 1 through 4, wherein the patterned coating layer has a coating application thickness that is varied across the raised profile over the electrode base surface.

### EXAMPLE 7

The surgical instrument of Example 6, wherein the patterned coating layer has a first coating application thickness at a distal end of the electrode base surface and a second coating application thickness at a proximal end of the electrode base surface, and wherein the second coating application thickness is greater than the first coating application thickness.

### EXAMPLE 8

The surgical instrument of Example 6, wherein the patterned coating layer has a first coating application thickness at a distal end of the electrode base surface and a second coating application thickness at a proximal end of the electrode base surface, and wherein the first coating application thickness is greater than the second coating application thickness.

### EXAMPLE 9

The surgical instrument of one or more of Examples 1 through 8, wherein the coating material is applied to the electrode base surface directly into the patterned coating layer by a first application method and wherein the first application method includes at least one of injection molding, insert molding, overmolding, lamination, screen printing, die bonding, spray coating, dip coating, spin coating, or additive manufacturing.

### EXAMPLE 10

The surgical instrument of one or more of Examples 1 through 8, wherein the coating material is first applied to an entirety of the electrode base surface and then a plurality of portions of the coating material are selectively removed by a selective removal method to form the plurality of openings in the patterned coating layer.

### EXAMPLE 11

The surgical instrument of Example 10, wherein the selective removal method includes at least one of laser ablation, photolithographic removal, acid etching, chemical etching, plasma etching, or abrasive spraying.

### EXAMPLE 12

The surgical instrument of Example 10, wherein the plurality of openings each have a removal depth of less than or equal to a coating application thickness of the coating material.

### EXAMPLE 13

The surgical instrument The surgical instrument of one or more of Examples 1 through 12, wherein the patterned coating layer is a crisscross patterned coating layer, or a honeycomb patterned coating layer.

### EXAMPLE 14

The surgical instrument of one or more of Examples 1 through 13, wherein the end effector further includes an ultrasonic blade, and wherein the electrode is incorporated into the ultrasonic blade.

### EXAMPLE 15

A surgical instrument comprising: a shaft assembly; an end effector extending distally from the shaft assembly; a first jaw of the end effector with an electrode configured to apply energy to a tissue of a patient, the electrode comprising: an electrode base surface, and a patterned coating layer having a coating material on the electrode base surface defining a raised profile offset from the electrode base surface, wherein the patterned coating layer further defines a plurality of openings to thereby expose a plurality of bare electrode portions of the electrode base surface therethrough; a second jaw of the end effector with an electrode configured to apply energy to a tissue of a patient, the electrode comprising: an electrode base surface; and a patterned coating layer having a coating material on the electrode base surface defining a raised profile offset from the electrode base surface, wherein the patterned coating layer further defines a plurality of openings to thereby expose a plurality of bare electrode portions of the electrode base surface therethrough; wherein the raised profile of the patterned coating layer of the first jaw has a first coating application thickness at a distal end of the electrode base surface and a second coating application thickness at a proximal end of the electrode base surface, and wherein the second coating application thickness is greater than the first coating application thickness, wherein the raised profile of the patterned coating layer of the second jaw has a first coating application thickness at a distal end of the electrode base surface and a second coating application thickness at a proximal end of the electrode base surface, and wherein the second coating application thickness is greater than the first coating application thickness, wherein a coverage ratio of the raised profile of the patterned coating layer to the plurality of bare electrode portions of the electrode base surface of the first jaw is between about 15% to about 75% of a total surface area of the electrode base surface, and wherein a coverage ratio of the raised profile of the patterned coating layer to the plurality of bare electrode portions of the electrode base surface of the second jaw is between about 15% to about 75% of a total surface area of the electrode base surface.

### EXAMPLE 16

A method of manufacturing a surgical instrument that includes an electrode configured to apply energy to a tissue of a patient, the method comprising: applying a coating material that forms a patterned coating layer located on an electrode base surface of the electrode to form a raised profile offset from the electrode base surface; wherein the patterned coating layer further defines a plurality of openings to thereby expose a plurality of bare electrode portions of the electrode base surface therethrough; and wherein a coverage ratio of the raised profile of the patterned coating layer to the plurality of bare electrode portions of the electrode base surface is between about 15% to about 75% of a total surface area of the electrode base surface.

### EXAMPLE 17

The method of Example 16, wherein the patterned coating layer is a crisscross patterned coating layer, or a honeycomb patterned coating layer.

### EXAMPLE 18

The method of Example 16, wherein the coating material is applied to the electrode base surface directly into the patterned coating layer by a first application method and wherein the first application method includes at least one of injection molding, insert molding, overmolding, lamination, screen printing, die bonding, spray coating, dip coating, spin coating, or additive manufacturing.

### EXAMPLE 19

The method of Example 16, wherein the coating material is first applied to an entirety of the electrode base surface and then a plurality of portions of the coating material are selectively removed by a selective removal method to form the plurality of openings in the patterned coating layer.

### EXAMPLE 20

The method of Example 19, wherein the selective removal method includes at least one or laser ablation, photolithographic removal, acid etching, chemical etching, plasma etching, or abrasive spraying.

### IV. Miscellaneous

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

It should also be understood that any ranges of values referred to herein should be read to include the upper and lower boundaries of such ranges. For instance, a range expressed as ranging “between approximately 1.0 inches and approximately 1.5 inches” should be read to include approximately 1.0 inches and approximately 1.5 inches, in addition to including the values between those upper and lower boundaries.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical instrument, comprising:
(a) a shaft assembly; and
(b) an end effector extending distally from the shaft assembly, wherein the end effector includes an electrode configured to apply energy to a tissue of a patient, the electrode comprising:
(i) an electrode base surface, and
(ii) a patterned coating layer having a coating material on the electrode base surface defining a raised profile offset from the electrode base surface, wherein the patterned coating layer further defines a plurality of openings to thereby expose a plurality of bare electrode portions of the electrode base surface therethrough,
wherein a coverage ratio of the raised profile of the patterned coating layer to the plurality of bare electrode portions of the electrode base surface is between about 15% to about 75% of a total surface area of the electrode base surface.

2. The surgical instrument of claim 1, wherein the coating material includes a dielectric material.

3. The surgical instrument of claim 2, wherein the dielectric material includes at least one of a plastics, a ceramic, a polymer, or a glass material.

4. The surgical instrument of claim 1, wherein the patterned coating layer includes a coating application thickness of between about 150 nm and about 700 nm.

5. The surgical instrument of claim 1, wherein the patterned coating layer has a coating application thickness that is consistent across the entirety of the raised profile over the electrode base surface.

6. The surgical instrument of claim 1, wherein the patterned coating layer has a coating application thickness that is varied across the raised profile over the electrode base surface.

7. The surgical instrument of claim 6, wherein the patterned coating layer has a first coating application thickness at a distal end of the electrode base surface and a second coating application thickness at a proximal end of the electrode base surface, and wherein the second coating application thickness is greater than the first coating application thickness.

8. The surgical instrument of claim 6, wherein the patterned coating layer has a first coating application thickness at a distal end of the electrode base surface and a second coating application thickness at a proximal end of the electrode base surface, and wherein the first coating application thickness is greater than the second coating application thickness.

9. The surgical instrument of claim 1, wherein the coating material is applied to the electrode base surface directly into the patterned coating layer by a first application method and wherein the first application method includes at least one of injection molding, insert molding, overmolding, lamination, screen printing, die bonding, spray coating, dip coating, spin coating, or additive manufacturing.

10. The surgical instrument of claim 1, wherein the coating material is first applied to an entirety of the electrode base surface and then a plurality of portions of the coating material are selectively removed by a selective removal method to form the plurality of openings in the patterned coating layer.

11. The surgical instrument of claim 10, wherein the selective removal method includes at least one of laser ablation, photolithographic removal, acid etching, chemical etching, plasma etching, or abrasive spraying.

12. The surgical instrument of claim 10, wherein the plurality of openings each have a removal depth of less than or equal to a coating application thickness of the coating material.

13. The surgical instrument of claim 1, wherein the patterned coating layer is a crisscross patterned coating layer, or a honeycomb patterned coating layer.

14. The surgical instrument of claim 1, wherein the end effector further includes an ultrasonic blade, and wherein the electrode is incorporated into the ultrasonic blade.

15. A surgical instrument comprising:
(a) a shaft assembly;
(b) an end effector extending distally from the shaft assembly;
(c) a first jaw of the end effector with an electrode configured to apply energy to a tissue of a patient, the electrode comprising:
(i) an electrode base surface, and
(ii) a patterned coating layer having a coating material on the electrode base surface defining a raised profile offset from the electrode base surface, wherein the patterned coating layer further defines a plurality of openings to thereby expose a plurality of bare electrode portions of the electrode base surface therethrough;
(d) a second jaw of the end effector with an electrode configured to apply energy to a tissue of a patient, the electrode comprising:
(i) an electrode base surface, and
(ii) a patterned coating layer having a coating material on the electrode base surface defining a raised profile offset from the electrode base surface, wherein the patterned coating layer further defines a plurality of openings to thereby expose a plurality of bare electrode portions of the electrode base surface therethrough;
wherein the raised profile of the patterned coating layer of the first jaw has a first coating application thickness at a distal end of the electrode base surface and a second coating application thickness at a proximal end of the electrode base surface, and wherein the second coating application thickness is greater than the first coating application thickness,
wherein the raised profile of the patterned coating layer of the second jaw has a first coating application thickness at a distal end of the electrode base surface and a second coating application thickness at a proximal end of the electrode base surface, and wherein the second coating application thickness is greater than the first coating application thickness,
wherein a coverage ratio of the raised profile of the patterned coating layer to the plurality of bare electrode portions of the electrode base surface of the first jaw is between about 15% to about 75% of a total surface area of the electrode base surface, and
wherein a coverage ratio of the raised profile of the patterned coating layer to the plurality of bare electrode portions of the electrode base surface of the second jaw is between about 15% to about 75% of a total surface area of the electrode base surface.

16. A method of manufacturing a surgical instrument that includes an electrode configured to apply energy to a tissue of a patient, the method comprising:
(a) applying a coating material that forms a patterned coating layer located on an electrode base surface of the electrode to form a raised profile offset from the electrode base surface;
wherein the patterned coating layer further defines a plurality of openings to thereby expose a plurality of bare electrode portions of the electrode base surface therethrough; and
wherein a coverage ratio of the raised profile of the patterned coating layer to the plurality of bare electrode portions of the electrode base surface is between about 15% to about 75% of a total surface area of the electrode base surface.

17. The method of claim 16, wherein the patterned coating layer is a crisscross patterned coating layer, or a honeycomb patterned coating layer.

18. The method of claim 16, wherein the coating material is applied to the electrode base surface directly into the patterned coating layer by a first application method and wherein the first application method includes at least one of injection molding, insert molding, overmolding, lamination, screen printing, die bonding, spray coating, dip coating, spin coating, or additive manufacturing.

19. The method of claim 16, wherein the coating material is first applied to an entirety of the electrode base surface and then a plurality of portions of the coating material are selectively removed by a selective removal method to form the plurality of openings in the patterned coating layer.

20. The method of claim 19, wherein the selective removal method includes at least one or laser ablation, photolithographic removal, acid etching, chemical etching, plasma etching, or abrasive spraying.
